(19) United States
(12) Patent Application Publication
(10) Pub. No.: US 2026/0232344 A1
(43) Pub. Date: Aug. 13, 2026

(54) AUTOMATED VASCULAR ACCESS DEVICE WITH INTEGRATED IMAGING AND OPTICAL DETECTION SYSTEM

(71) Applicants: UNIVERSITY OF MARYLAND, BALTIMORE, Baltimore, MD (US); MDC STUDIO, INC., Baltimore, MD (US)

(72) Inventors: Rajabrata Sarkar, Ellicott City, MD (US); Khanjan Nagarsheth, Ellicott City, MD (US); Jeffrey Gair, Jr., Monrovia, MD (US)

(21) Appl. No.: 19/533,925

(22) Filed: Feb. 9, 2026

Related U.S. Application Data
(60) Provisional application No. 63/755,540, filed on Feb. 7, 2025.

(51) Int. Cl. A61B 17/34; A61B 8/00; A61B 8/08

(52) U.S. Cl. CPC A61B 17/34; A61B 8/0891; A61B 8/461; A61B 8/488; A61B 2017/3413

(57) ABSTRACT

An automated vascular access device integrates ultrasound imaging with trajectory guidance for simplified central venous and arterial cannulation. The device includes a software-generated crosshair superimposed on real-time ultrasound images that indicates predicted needle trajectory. An operator adjusts the crosshair position via thumb control, and a linear servo actuator automatically tilts a needle carriage to align a needle trajectory with the crosshair location. The device incorporates a needle-dilator having variable stiffness elements formed by scalloped indentations and relaxing incisions, allowing sufficient rigidity for vessel puncture while permitting flexibility for advancement. An optical guidewire detection system uses laser beams, photocell detectors, and mirrors to track guidewire position and advancement distance while maintaining sterility. Additional features include color-flow Doppler for artery-vein differentiation, auto-retracting skin lancet, and multi-modal sensing with MEMS-based pressure transducers and bioimpedance sensors. The device enables less experienced providers to perform vascular access procedures with increased safety and success rates.

### CROSS-REFERENCE TO RELATED APPLICATIONS

This application claims priority from U.S. Provisional Patent Application No. 63/755,540 titled “Automated Emergency Femoral Artery Sheath Placement Device” filed with the United States Patent and Trademark Office on Feb. 7, 2025, the specification of which is incorporated herein by reference in its entirety.

### FIELD OF THE INVENTION

The present invention generally relates to the fields of cardiovascular and emergency medicine. More specifically, the present invention relates to an automated vascular access device with integrated ultrasound imaging, optical guidewire detection, and variable stiffness needle-dilator components for central venous and arterial cannulation.

### BACKGROUND

Arterial cannulation is used widely in the clinical management of critically ill adults, with arterial circulatory invasion second in frequency only to intravenous cannulation. It provides an uninterrupted display of pulse contour and continuous beat-to-beat hemodynamic measurement. This data can be invaluable for effective clinical management, such as the reliable titration of supportive medications. Numerous patient conditions, including morbid obesity, burn extremities and shock can cause non-invasive blood pressure measurements to be inaccurate and so necessitate invasive blood pressure monitoring. The procedure of arterial cannulation comes with some risk and so the need must be weighed against the risk to the patient. Arterial cannulation is performed on a number of vessels including the radial, femoral, axillary, brachial, ulnar, dorsal pedis, tibial posterior and temporal arteries.

Femoral artery cannulation has numerous advantages over cannulation of other sites. Femoral cannulation provides a pulse contour approximating aortic with minimal thrombotic risk. There is little evidence to show increased incidence of catheter-related systemic infection at this site.

The femoral artery lies in a neurovascular bundle lateral to the femoral vein and median to the femoral nerve (as seen in FIG. 1). The femoral artery is palpated midway between the anterosuperior iliac spine and the symphysis pubis. Collateral circulation exists via a number of anastomoses, and the large vessel diameter allows catheter longevity twice that of radial catheters. Prospective and retrospective studies detail the relative safety of this site for hemodynamic monitoring. A potential exists, however, for extraperitoneal hemorrhage, vascular injury from common branch entry, and cannulation hematoma. Femoral artery catheter complications, though infrequent, are complicated, difficult to identify, and may be associated with significant mortality. The femoral artery usually can be cannulated, even during profound shock states.

An application for the procedure is emergent or urgent cannulation of the femoral artery for subsequent placement of a REBOA balloon or intraaortic balloon pump. REBOA is Resuscitative Endovascular Balloon Occlusion of the Aorta and is a lifesaving device for use in patients with pelvic fractures, penetrating injuries, life threatening hemorrhage, ruptured abdominal aortic aneurysms, and other emergency conditions. REBOA may have the greatest benefit when deployed early, and has been applied in the field (pre-ambulance) in Europe. There is clear military importance in deployment of REBOA in the field. An intra-aortic balloon pump is used to support patients in cardiogenic shock, and is also often deployed under urgent or emergent conditions.

Femoral artery cannulation is a valuable procedure, but at present clinicians with advanced training must perform the procedure. Cannulation and sheath placement in the femoral artery currently requires a physician with advanced training (vascular surgery, trauma surgery, interventional radiology or interventional cardiology) and involves multiple needle, scalpel and wire exchanges (Seldinger technique, see FIG. 2). In an emergency setting, the femoral pulse may be absent or decreased due to hypotension which further complicates accurate localization for cannulation.

Central venous cannulation is common, required by 80% of all ICU patients, but is a skill that remains challenging for practitioners to learn and safely execute. Because it's difficult and patient anatomy is variable, significant complications and technical failures are frequent. The clinical need for a product that simplifies cannulation is large.

Central venous catheterization is a life-saving procedure commonly performed on critically ill patients. This requires needle puncture and catheterization of a central vein using the Seldinger technique. Ultrasound guidance for central venous puncture has reduced complication rates, but requires skill in vessel localization, visualizing the needle's tip, and a series of coordinated two-handed maneuvers. Central venous cannulation has higher success rates when performed by more experienced physicians but is often done emergently by available less experienced physicians. Despite decades of external ultrasound assistance, these procedures remain associated with 5-19% rates of complication and failed puncture even in the hands of experienced operators using ultrasound guidance. In a large, randomized trial of ultrasound guidance for femoral artery puncture, ultrasound decreased complications but did not increase procedural success, emphasizing the persistent pitfalls of this procedure even with highly trained physicians with imaging guidance. Thus, there is a critical unmet need for a portable, self-contained semi-automated device to make central venous (and arterial) cannulation safer and faster in the hands of less experienced providers.

Because of its complexity, the Seldinger procedure is expensive, typically billed to third party payers at $1,100. Device manufacturers provide sterile single-use packs for internal jugular vein. A standard Arrow Inc. central line kit ranges in cost from $273-$1,414 depending on catheter size and configuration. Medicare recently elected to no longer reimburse secondary costs of inpatient complications with private insurers likely to follow. Thus, hospitals have a strong financial incentive to prevent common and serious complications (pneumothorax, hemorrhage, arterial injury or thrombosis).

The incidence of these procedures is increasing substantially. Central venous (and arterial) cannulation is routinely guided by ultrasound but remains difficult in terms of procedural success and has a persistent high incidence (15-20%) of serious complications. Typical devices end with wire insertion, which is dependent on the operator to push the wire forward. Thus, no typical devices complete the Seldinger procedure, and none end the procedure with an actual catheter in the blood vessel. Typical devices require additional bimanual steps by the operator to complete the procedure. This requires that the operator establish a sterile field, don sterile gloves, and complete the procedure with additional components that can reduce the likelihood for adoption by less experienced non-specialist providers. This is particularly true given the potential for serious errors with the bimanual exchange, including: 1) inadvertent removal of the guidewire while retracting the needle, 2) inadvertent loss of the guidewire inside the patient due to forward motion of the guidewire during sheath/dilator advancement, and 3) dislodging the sheath from within the target vessel when retracting the guidewire and dilator.

Earlier automated vascular access devices have been developed to address some of the foregoing challenges. U.S. Pat. No. 10,702,676 to Sarkar et al. discloses an automated emergency arterial sheath placement device having a body with a handle and an actuator, and an arterial sheath placement head configured for placement against a patient's skin. The device includes a Doppler sensor for artery localization and automated sequential advancement of an arterial needle, guide wire, and arterial sheath. Similarly, U.S. Pat. No. 11,628,274 to Sarkar et al. (a continuation of the '676 patent) discloses related automated arterial sheath placement systems using Doppler technology. While these prior devices represent significant advances in automating the Seldinger technique, they rely solely on Doppler sensing without real-time ultrasound imaging guidance, utilize separate needle and dilator components, and lack optical detection systems for precise guidewire tracking. The present invention addresses these limitations through integration of commercial ultrasound units with trajectory guidance, integrated needle-dilator components with variable stiffness characteristics, and novel optical guidewire detection systems.

Despite advancements in the technological landscape, there remains a significant need to solve the clinical problems left by existing devices. The current solutions do not address the challenges of performing central venous and arterial cannulation in pre-hospital settings or by less experienced providers. The proposed invention aims to fill this gap by providing a portable, self-contained, semi-automated device that simplifies the procedure, reduces complications, and makes it accessible to a broader range of healthcare providers.

The above information disclosed in this Background section is only for enhancement of understanding of the background of the invention and therefore it may contain information that does not form any part of the prior art.

### SUMMARY

Provided according to many embodiments is an Automated Vascular Access Device that integrates sensors and drivers to enable a cannula to be precisely placed in the internal jugular vein. The device addresses the critical unmet need of enabling a non-expert provider to accurately and safely place a sheath in the internal jugular vein (follow-on indications will include the femoral vein and artery). Some embodiments are configured with automated needle advancement, sensing of arterial vs. venous puncture, automated guidewire advance, and integration with point-of-care ultrasound. One embodiment of the device comprises a hand-held, self-contained, battery-powered device that identifies and automatically punctures and cannulates a major vessel. The device can be used by medics in the field, as well as in-hospital. Its advanced sensing technology and automation will substitute for the tactile and haptic cues that experienced vascular surgeons rely on to accomplish cannulation.

In one embodiment, a compact, battery-powered device is configured to address several technical errors, including: 1) moving the ultrasound probe while focusing on the needle puncture, 2) imaging only the artery or the needle tip in the 2-D plane of the ultrasound, 3) miscalculating the needle's trajectory (jugular vein depth is surprisingly variable), and 4) dislodging the needle tip during advancement of the guidewire. Some embodiments of the device include at least one of a) a rigidly mounted needle guide and ultrasound probe to reduce stray movement, b) guidance for the needle's trajectory, c) automated needle and guidewire advancement, and d) automated confirmation of successful puncture and advancement of the guidewire.

The device includes a handle that accepts a self-contained commercial handheld ultrasound unit and an exemplary configuration can be configured with commercially-available units (e.g., Butterfly IQ+unit) and a mobile device-based screen and user interface. The software architecture has flexibility to incorporate handheld ultrasound units from various manufacturers (e.g., Philips).

At least one embodiment of the device may offer one or more advantages over typical devices and manual kits. For example, using built-in vessel imaging and automated sensing of vessel puncture, this device may allow resident physicians, P.A. s, advanced practice nurses, and paramedics to more effectively and confidently practice this life-saving technique. As a further example, by automating needle advancement and guidewire control, the device may decrease the failure rate and complication rate of these common procedures, compared to typical devices. Thus, hospital costs and more importantly patient safety may be favorably affected, compared to typical devices. The increased emphasis by health care payors and systems on value-based care may also drive investment in the device as the avoidable costs and increased length of stay of complications will adversely affect value-based care assessments.

In some embodiments, the device is configured to allow imaging devices (e.g., Butterfly IQ+) to be positioned so that the plane of the image is perpendicular to the vessel, and centered at the point of insertion. It can further be at a fixed distance from the point of insertion.

In other embodiments, the device can include an echogenic needle (~8 cm long) attached to a custom needle hub containing several sensors. The angle of attack will be adjustable in a range of angles, such as from 30° to 70°, with respect to the plane of the skin. In some embodiments, the needle is advanced through the skin using an actuator, such as a linear servo actuator, with real-time positional feedback. Ultrasound imaging can track the needle as it approaches the target vessel, and the hub's sensors will detect entry into the lumen through a “flash” of blood (e.g., optical sensing; hydrostatic pressure sensing via MEMS-based transducer, bioimpedance sensing, and the like). At least one embodiment of the device includes a needle delivery system that can be built from off-the-shelf components and custom stereolithography-based 3D printed parts.

In one embodiment, the device includes platform-mounted capstans configured to drive a 0.035″ wire that will be advanced through the barrel of the needle, and 10 cm into the vessel. The device is further configured for more precise control of guidewire tip position, compared to typical devices. In one embodiment, the device includes capstans configured to grip the guidewire firmly (with minimal slippage). Each capstan can have position sensors, such as servomotors or rotary encoders. For example, a first capstan is directly controlled by a digitally-controlled servomotor, while a second capstan has a rotary encoder mounted to track wire advancement. As a further example, the device confirms guidewire advancement without slippage using signals from the capstans'position sensors. In another embodiment, the device integrates motors, drivers, and sensors via low-level communication to a microcontroller, such as an Arduino. Furthermore, synthesized data can then be delivered from the Arduino to an application running on an Android OS tablet.

In one embodiment of the device, the position of the needle tip can be displayed on an ultrasound image. The API output of the Butterfly IQ+ can be exported to a tablet, where a second API showing the calculated position of the needle tip may be superimposed. This will set the stage for the operator to change the reticle position, with that command being transmitted to the device to advance or retract the needle.

Proof-of-concept tests of the device were carried out on a test bed (synthetic tissue and vessels) by cannulating a simulated internal jugular vein, though in other examples, the device can be used in other vessels, such as a femoral vein. The operator uses, for example, Butterfly IQ+ images as displayed on the tablet to set the needle's angle of attack and direct its advance. In one test, success will be defined as penetration of the vessel lumen followed by guidewire entry into the lumen, for example in at least 6 of 10 tries.

According to other embodiments, the device can be configured for integration of color flow ultrasound into an automated vascular access puncture device. This intervention allows the user of the device to visualize either arteries or veins. It allows the user to identify and distinguish arteries from veins using the color-flow feature that is present on most self-contained hand-held ultrasound units. The intervention includes the integration of the needle trajectory for the puncture with the simultaneous ultrasound image. The needle trajectory is derived from the tilting of the automated needle carriage that is part of the automated vascular access device. The needle trajectory is semi-automated, in that the user with a thumb control can adjust the needle trajectory prior to puncture up and down based on the ultrasound image.

This intervention allows a crosshair or reticle to be generated, such as by software. This crosshair symbol is moved up and down on the ultrasound screen by a thumb control on the handle of the automated vascular access device, and is superimposed on the real-time ultrasound image generated at the front of the device. The crosshair indicates where the needle will progress forward should the user choose to activate needle puncture at that position. The user may identify a blood vessel on color-flow duplex imaging and confirm that the signal was either arterial or venous. The user may then adjust with the thumb control the crosshair up and down to center it over the middle of the vessel. In doing so the device automatically tilts the needle carriage to the corresponding angle to intersect the crosshair point in the plane of the ultrasound image. Should the user then pull the trigger to activate needle puncture, the device advances the needle at that angle and intersects the plane of the ultrasound at the point indicated by the crosshair. Actual puncture of the vessel would be accomplished by sensing return of blood within the needle and also determining its pulsatility and pressure.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE FIGURES

The accompanying drawings, which are included to provide a further understanding of the invention and are incorporated in and constitute a part of this specification, illustrate embodiments of the invention, and together with the description serve to explain the principles of the invention.

FIG. 1 shows a schematic view of a femoral artery lying in a neurovascular bundle lateral to the femoral vein and median to the femoral nerve.

FIG. 2 shows the steps involved in performing cannulation and sheath placement in the femoral artery according to the Seldinger technique.

FIG. 3(a) shows schematically a side view of an Automated Emergency Femoral Artery Sheath Placement Device according to an exemplary embodiment of the invention.

FIG. 3(b) shows schematically a front view of the head of an Automated Emergency Femoral Artery Sheath Placement Device according to an exemplary embodiment of the invention.

FIG. 4(a) shows a side view schematic of the needle/wire/sheath unit, according to an exemplary embodiment of the invention. Advancement of the sheath may detach a side-mounted pressure transducer from the side of the needle.

FIG. 4(b) shows a top view schematic of the scalpel, the gel/antiseptic needle and the anesthetic needles arrangement relative to the arterial entry needle, according to an exemplary embodiment of the invention.

FIG. 5(a) shows a schematic diagram depicting a side view of the position of the device, while in use, with respect to the thigh and the femoral artery according to an exemplary embodiment of the invention.

FIG. 5(b) shows a schematic diagram depicting a front view of the position of the device, while in use, with respect to the femoral artery according to an exemplary embodiment of the invention.

FIG. 6(a) shows schematically a front view of the head of an Automated Emergency Femoral Artery Sheath Placement Device according to another exemplary embodiment of the invention.

FIG. 6(b) shows schematically a cross-section view, through cross-section “S” in FIG. 6(a), of the head of an Automated Emergency Femoral Artery Sheath Placement Device according to another exemplary embodiment of the invention.

FIG. 7 shows a flowchart depicting the steps of a method for using the automated emergency femoral artery sheath placement device, according to an exemplary embodiment of the invention.

FIG. 8 shows a schematic diagram of an integrated needle-dilator with variable stiffness features including scalloped indentations and relaxing incisions, according to an exemplary embodiment of the invention.

FIG. 9 shows a schematic diagram of an ultrasound-guided trajectory system with crosshair guidance interface superimposed on real-time ultrasound imaging and adjustable needle trajectory mechanism, according to an exemplary embodiment of the invention.

FIG. 10 shows a schematic diagram of an optical guidewire detection system using laser beam paths, photocell detectors, and mirrors to maintain sterility, according to an exemplary embodiment of the invention.

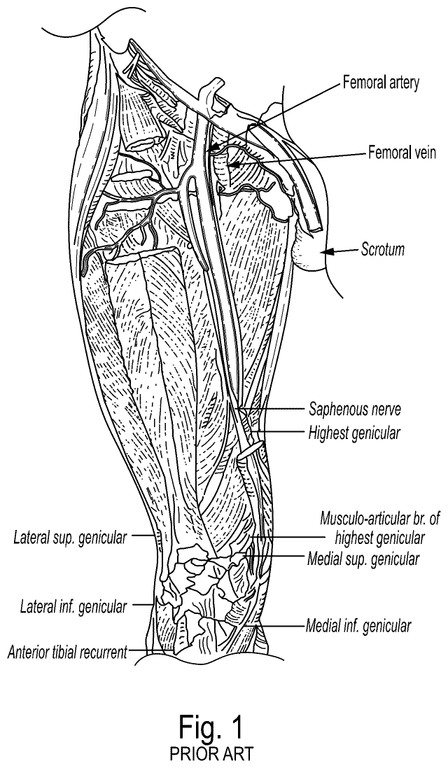

### DETAILED DESCRIPTION OF THE PRESENT INVENTION

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

Hereinafter, an automated vascular access device and method is disclosed. This invention may, however, be embodied in many different forms and should not be construed as limited to the exemplary embodiments set forth herein. Rather, these exemplary embodiments are provided so that this disclosure is thorough, and will fully convey the scope of the invention to those skilled in the art.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

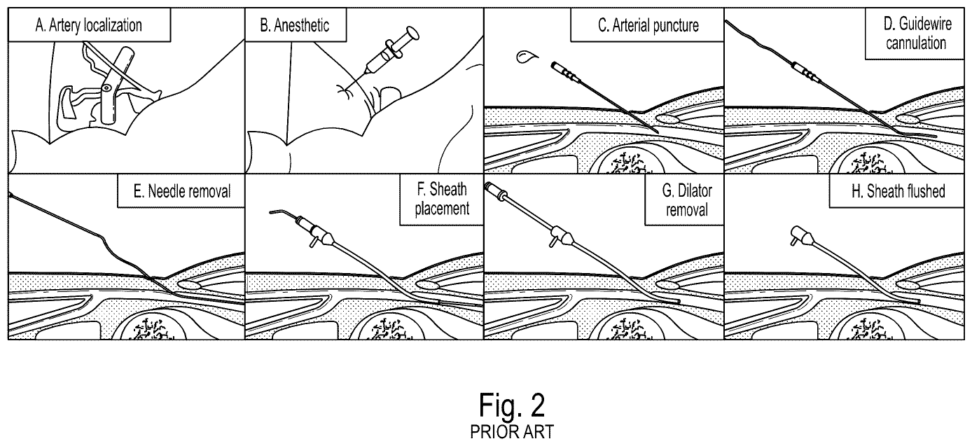

The disclosure herein presents a portable (and optionally battery-powered) device for vascular cannulation that integrates ultrasound imaging technology with automated components to perform all steps of the Seldinger technique (FIG. 2). The device is referred to hereinafter as an Automated Vascular Access Device (AVAD) or Automated Emergency Femoral Artery Sheath Placement Device (eFASP). No such devices are available to allow emergency providers (physicians or non-physicians) without specialized training to obtain rapid vascular access and sheath placement in an emergency or pre-hospital setting with integrated ultrasound guidance and optical guidewire detection. Additional applications of this device may include:
1. Urgent placement of a central venous or arterial sheath for blood pressure monitoring and blood gas sampling in the emergency room; hypotensive patients requiring pressor therapy or going to procedures involving invasive arterial line placement. This invention could rapidly facilitate this in critically ill and/or hypotensive patients.
2. Accurate cannulation of the internal jugular vein for central venous access by emergency room personnel, paramedics, or nurses in pre-hospital settings.
3. Accurate cannulation of the femoral artery by emergency room personnel in preparation for cardiac catheterization. Acute myocardial infarction patients require emergent cardiac catheterization where the interventional cardiologist accesses the femoral artery as the first step. This device could allow emergency room or cath lab personnel to pre-place the sheath and potentially decrease “door to balloon” times.
4. Variants of this device would permit safer and easier radial and brachial artery cannulation. Access of the radial artery for blood pressure monitoring and blood gas sampling is commonly done in the operating room and ICU, as is brachial artery access. Both are associated with a high rate of failure and potentially significant complications. This represents an additional market opportunity for the proposed device.

The Automated Vascular Access Device (AVAD or eFASP) is designed to allow emergency medical providers without specialized vascular training to rapidly and accurately obtain vascular sheath access under emergency conditions with integrated ultrasound guidance. The device is self-contained and does not require external wires, scalpels, or additional imaging equipment, which are all currently required for conventional sheath placement.

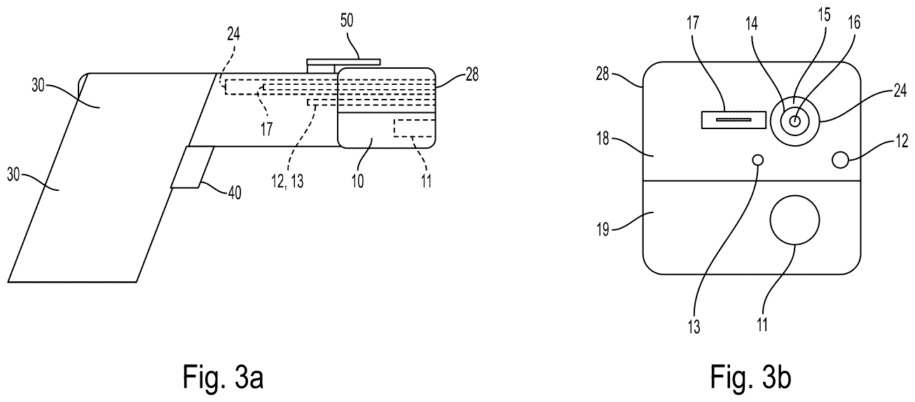

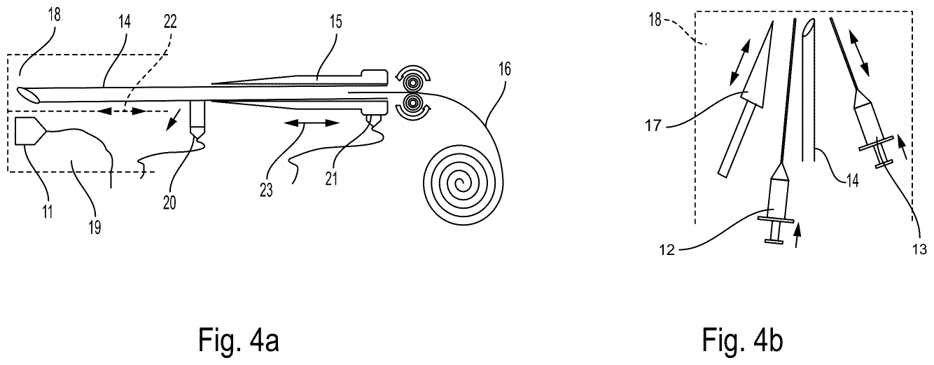

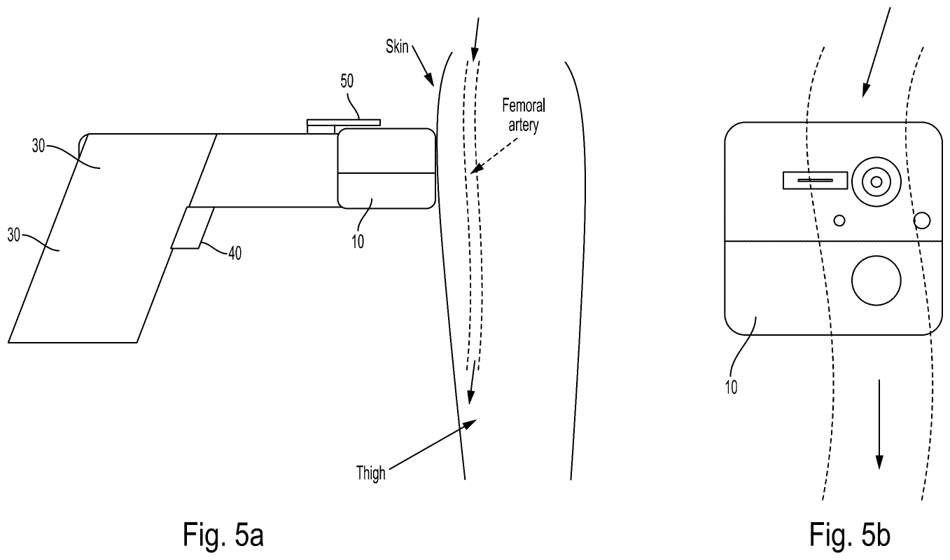

Vascular cannulation involves multiple needles, scalpel, wire exchanges, and sheaths as shown in FIG. 2 (Seldinger technique) and FIG. 4(a) and 4(b). The device disclosed herein incorporates such components into a single compact and easy to operate device. FIG. 3(a) shows a side view of an AVAD device according to an exemplary embodiment of the invention. The AVAD device may include a device head 10, a body 30, a trigger 40, an LCD display screen 50, and a battery (not shown). The AVAD is configured such that an operator holds the body part 30 and actuates the trigger 40 by his/her finger in a manner similar to operating a cordless drill.

FIG. 3(b) shows a front view of the device head 10. The device head 10 may include a Doppler sensor 11 and associated electronic components, a needle 12 for releasing Doppler gel and antiseptic; a needle 13 for injecting anesthetic, an arterial entry needle 14, a sheath 15, and a guide wire 16, all of which are extensible through port 24; and a scalpel 17. The device head may include a first unit 18 and a control unit 19. The unit 18 may include the needles 12-14, the sheath 15, the guide wire 16, and the scalpel 17. The arterial entry needle 14, the sheath 15 and the guide wire 16 may be disposed concentrically as shown by FIG. 3(b).

According to exemplary embodiments of the invention the sizes of the device head's components and the distances between such components may be as follows. The sheath 15 may be about 3-4 mm in diameter or may be 7 French (approximately 2.3 mm diameter). The arterial needle 14 may have a diameter which is about 1 mm smaller than the diameter of sheath 15. The guide wire 16 may be about 2 mm in diameter or may be 0.035 inches in diameter. The Doppler sensor 11 may be about 10 mm in diameter.

The arterial needle 14 may be about 12 inches long and may have a motion range of about 3 inches. The arterial sheath 15 may be about 4 inches long. The wire 16 may be about 36 inches long. The range of motion for the sheath may be about 4 inches (it will be almost completely within the patient at the end of the procedure). The range of motion for the wire may be about 9 inches forward. The scalpel 17 may be a size #11 blade or may be about 4 mm wide and about 10 mm long.

The distance between the centers of Doppler sensor 11 and the sheath 15 may be about 3 cm. The distance between the blade 17 and sensor 11 may be approximately 5 cm between the centers of each. The distance between blade 17 and wire 16 may be about 4 cm between the centers of each. The two sides of the device head 10 shown in FIG. 3(b) may be about 6 cm long. The average common femoral artery is about 12 mm in diameter and approximately 3-6 cm below the skin.

It will be understood by those skilled in the art that the above dimensions are provided only as exemplary and various changes in form and details may be made thereto without departing from the spirit and scope of the present disclosure as defined by the appended claims.

The AVAD may be designed such that the two units 18 and 19 may be separated from each other. The first unit 18 may be disposable. The control unit 19 may include the Doppler sensor and the associated electronic components and may not be disposable. The two units 18 and 19 may be designed to lock together. The locking together of units 18 and 19 may be performed in either a completely sterile (operating room) or semi-sterile (pre-hospital use) manner. The AVAD resembles in size and configuration a cordless drill when docked.

FIG. 4(a) and (b) show exemplary embodiments of the type of components that may be incorporated in the device head 10 such as: the needle 12 for releasing Doppler gel and antiseptic, the needle 13 for injecting anesthetic, the arterial entry needle 14, and the scalpel 17. The AVAD may further include a first pressure transducer 20 configured to measure the pressure inside the lumen of the arterial needle 14 and a second pressure transducer 21 mounted on the sheath and configured to measure the pressure inside the lumen of the arterial sheath 15 (as seen in FIG. 4(a)). The first pressure transducer 20 is configured to break away upon moving/sliding the arterial sheath 15 over the arterial needle 14.

FIG. 4(a) shows the configuration and relative positions of the components 11 and 14-16 within the device head 10 and the body 30 according to an exemplary embodiment of the invention. FIG. 4(a) shows a side view schematic of needle/wire/sheath unit. Advancement of the sheath may detach a side-mounted pressure transducer 20 from the side of needle 14. Movements are shown by two head arrows. The mechanism (e.g. rack and pinion) driving the movements are omitted for clarity. As seen in FIG. 4(a), the arterial entry needle 14 is configured to slide inside the lumen of the arterial sheath 15 and conversely the arterial sheath 15 can slide over the arterial entry needle 14. The guide wire is configured to fit inside the lumen of the arterial entry needle such as to move along the entry needle. FIG. 4(b) shows a top view of the scalpel, the gel/antiseptic needle and the anesthetic needles arrangement relative to the arterial entry needle. The arrows indicate movement directions of these components. As seen in FIG. 4(b) the axes and the directions of movement of the scalpel, the entry needle, and the anesthetic needle may not be parallel (e.g., they may form an angle with respect to each other) such as to ensure that each of these surgical tools penetrate into the patient's skin at the right place and reach the desired location within the body of the patient with respect to the target vessel.

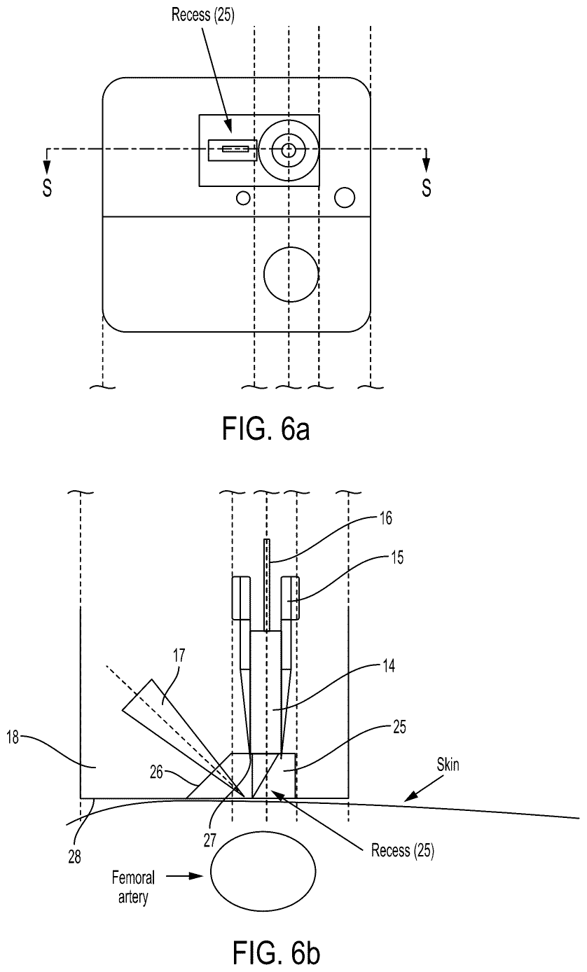

FIG. 6(a) and (b) show another exemplary embodiment of the invention. According to the embodiment in FIG. 6(a) and 6(b), the device head includes a recess 25 such as to enable the blade 17 to nick/cut the skin of the patient at the place where the arterial sheath 15 is configured to enter the skin. FIG. 6(a) shows a front view of the device head 10. FIG. 6(b) shows schematically a cross-section view, through cross-section “S” in FIG. 6(a), of the device head 10. FIG. 6(b) shows the positions of the components 14-17 with respect to each other and with respect to the patient's skin and target vessel.

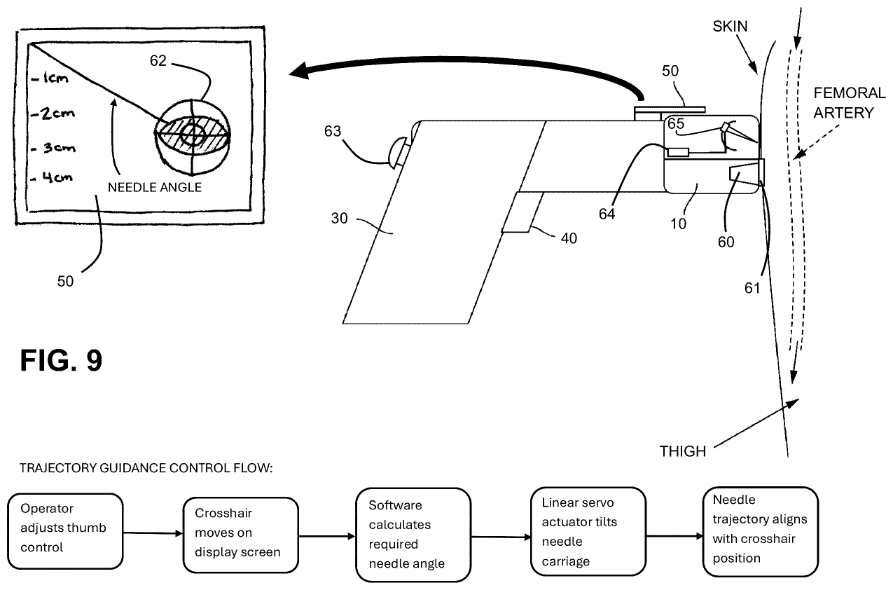

In a significant advancement over prior automated vascular access devices that rely solely on Doppler sensing (such as those disclosed in U.S. Pat. Nos. 10,702,676 and 11,628,274), in certain exemplary configurations the present device integrates a commercial handheld ultrasound unit into the device architecture. As shown in FIG. 9 (in which several of the above-described advancing components of device head 10 are omitted for clarity), the device mounts a commercially-available handheld ultrasound unit 60, such as the Butterfly IQ+ or Philips Lumify. The software architecture provides flexibility to integrate various manufacturers'ultrasound units through API integration. The ultrasound unit 60 is positioned at the device head 10 such that the ultrasound transducer 61 and its imaging plane is perpendicular to the target vessel and centered at the point of needle insertion, maintaining a fixed distance from the puncture site. This configuration allows real-time B-mode and color-flow Doppler imaging throughout the procedure.

The API output from the integrated ultrasound unit 60 (e.g., Butterfly IQ+) is exported to a display screen 50 (which may be an integrated or remotely connected tablet, smartphone, or dedicated display screen), where software overlays trajectory guidance information superimposed on the live ultrasound image. This integration addresses limitations in prior art systems by providing continuous visual feedback of both needle position and target vessel location throughout the procedure.

As illustrated in FIG. 9, the device incorporates a novel trajectory guidance system that integrates needle positioning with real-time ultrasound imaging. A software-generated crosshair or reticle 62 is superimposed on the live ultrasound image displayed on the screen 50. The crosshair 62 indicates the predicted needle trajectory and puncture location based on the current needle angle.

An operator control, such as a thumb control 63 positioned on the handle of the device body 30, allows the operator to adjust the crosshair position up and down on the ultrasound image. As the operator moves the crosshair 62 to center it over the target vessel visible on the ultrasound image, the device automatically adjusts the needle carriage 65 angle to correspond to the selected trajectory. A linear servo actuator 64 with real-time positional feedback mechanically tilts the needle carriage 65 through an adjustable range, typically from 30° to 70° with respect to the plane of the skin, to align the needle trajectory with the crosshair position 62 in the plane of the ultrasound image.

When the operator activates the trigger 40 to initiate needle advancement, the needle 14 (or integrated needle-dilator 14′ as described below) advances at the pre-selected angle and intersects the plane of the ultrasound image at the point indicated by the crosshair 62. This semi-automated trajectory adjustment system significantly reduces the technical skill required to accurately target vessels at varying depths, a persistent challenge in conventional ultrasound-guided procedures.

The integrated ultrasound system 60 includes color-flow duplex imaging capability that allows identification and differentiation of arteries from veins. The color-flow feature, present on most modern handheld ultrasound units, displays arterial flow in one color (typically red) and venous flow in another color (typically blue). This allows the operator to confirm vessel type before initiating puncture, ensuring that the correct vessel (arterial vs. venous) is targeted for the intended procedure. The pulsatility and pressure characteristics detected by sensors 20 and 21 provide additional confirmation of vessel type upon puncture.

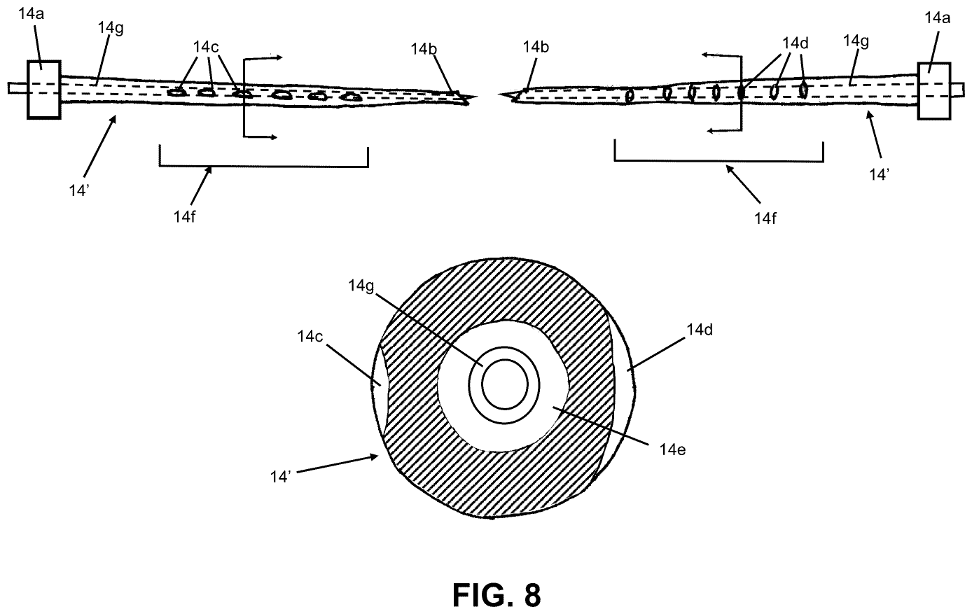

In a significant structural advancement over prior vascular access devices that utilize separate needle, dilator, and sheath components (as disclosed in U.S. Pat. Nos. 10,702,676 and 11,628,274), the present device incorporates an integrated needle-dilator 14′ component as shown in FIG. 8. The integrated needle-dilator 14′ combines the puncture function of a needle with the dilation function of a dilator into a single coaxial component, eliminating one exchange step in the Seldinger procedure and reducing the potential for complications.

The integrated needle-dilator 14′ is configured to be sufficiently rigid at its distal end 14b to puncture a patient's skin and vessel wall, while incorporating variable flexibility characteristics along its length to allow advancement over a curved guidewire. In one embodiment, the integrated needle-dilator 14′ is manufactured from polyetheretherketone (PEEK), a high-performance polymer that provides excellent strength, biocompatibility, and radiopacity. Manufacturing methods may include computer numerical control (CNC) milling or additive manufacturing techniques such as 3D printing.

The integrated needle-dilator 14′ features a conventional tapered design with an obliquely-cut tip at the distal end 14b for vessel puncture. A central lumen 14e extends from the proximal end 14a to the distal end 14b and is configured to receive a guidewire 16. To achieve variable stiffness characteristics, several structural features may be incorporated in the variable stiffness region 14f.

As shown in FIG. 8, scalloped indentations 14c may be formed on one side of the integrated needle-dilator 14′ along a portion of its length within the variable stiffness region 14f. These indentations, which may be semi-circular, oval, or other curved profiles, reduce the structural stiffness on that side of the device, allowing controlled bending in a predetermined direction when the device encounters resistance or must navigate a curved path.

Further, relaxing incisions 14d oriented orthogonal to the long axis of the dilator may be formed on the side opposite the bending direction (i.e., opposite the scalloped indentations 14c). These incisions 14d further facilitate controlled flexion of the integrated needle-dilator 14′.

In certain exemplary configurations, an integrated needle-dilator 14′ may incorporate both scalloped indentations 14c and relaxing incisions 14d to optimize variable stiffness characteristics. The depth, spacing, and configuration of these features can be tailored to achieve desired flexibility profiles for different clinical applications (e.g., internal jugular vein access vs. femoral artery access).

In some exemplary configurations, an absorbable suture (not shown) may be used to retain tension on the integrated needle-dilator 14′ during insertion, providing additional stiffness that gradually reduces as the suture is absorbed, allowing increased flexibility for sheath advancement.

The integrated needle-dilator 14′ may be sized as 7 French (approximately 2.3 mm outer diameter) or other clinically appropriate sizes. Testing in tissue block models with embedded vessels under physiologic pressures (arterial: 65 mm Hg; venous: 6 mm Hg) has demonstrated successful vessel puncture without posterior wall damage.

In certain exemplary embodiments, the needle-dilator 14′ achieves variable stiffness through a two-phase operational mechanism utilizing a central steel needle 14g positioned within the dilator lumen 14e. This embodiment provides dynamic stiffness control that varies between the puncture phase and the cannulation phase of the procedure.

During the puncture phase, a rigid central steel needle 14g is positioned concentrically within the lumen 14e of the integrated needle-dilator 14′. The steel needle 14g may extend substantially the full length of the integrated needle-dilator 14′ or may extend along a portion of its length sufficient to provide structural rigidity. The presence of the steel needle 14g within the dilator lumen 14e creates a composite structure in which the steel needle 14g provides an internal supportive core that stiffens the overall needle-dilator assembly. This stiffened configuration enables the integrated needle-dilator 14′ to penetrate the patient's skin and vessel wall with sufficient rigidity to avoid deflection or buckling. In this exemplary embodiment, the steel needle 14g may have an outer diameter ranging from approximately 18-gauge to 22-gauge (approximately 0.8 mm to 1.3 mm), while the integrated needle-dilator 14′ outer diameter may range from 5-French to 8-French (approximately 1.7 mm to 2.7 mm).

During the cannulation phase, after successful vessel puncture has been confirmed by the sensing systems described herein, the central steel needle 14g is constrained or held stationary while the dilator portion of the integrated needle-dilator 14′ continues to advance forward over the steel needle 14g and into the vessel. This differential advancement, wherein the steel needle 14g remains fixed while the dilator advances, effectively removes the internal structural support from the advancing portion of the dilator. As the dilator advances beyond the stationary steel needle tip, the unsupported dilator portion exhibits increased flexibility, allowing it to navigate curved anatomical paths, accommodate vessel angulation, and reduce the risk of posterior vessel wall puncture.

The transition from puncture phase to cannulation phase may be controlled by the device's control unit 19 through independent drive mechanisms. For example, a first drive mechanism (e.g., a linear actuator or rack and pinion system) may advance both the steel needle 14g and dilator together during the puncture phase. Upon detection of successful vessel entry by pressure transducers 20, bioimpedance sensors, or optical blood detection sensors, the control unit 19 may constrain the steel needle 14g in a fixed position while a second drive mechanism continues to advance the dilator and sheath 15 assembly forward over the now-stationary steel needle 14g.

In embodiments utilizing an integrated needle-dilator 14′ with variable stiffness features, the scalpel 17 described in the basic device configuration may be replaced with or configured as an auto-retracting spring-loaded lancet mechanism. The scalpel 17, as shown in FIG. 4(b) and 6(b), may be modified to function as an auto-retracting lancet configured to pre-puncture the patient's skin prior to advancement of the integrated needle-dilator 14′. In this enhanced configuration, the scalpel/lancet 17 comprises a spring-loaded steel blade positioned at the device head 10 that, upon trigger activation, rapidly advances to puncture approximately 3 mm of full-thickness skin, then automatically retracts. This pre-puncture reduces the force required for the integrated needle-dilator 14′ to traverse the skin, improving procedural success rates and reducing the risk of needle deflection or vessel movement during skin penetration.

In the original embodiment shown in FIG. 4(b) and 6(b), the scalpel 17 operates according to conventional timing (i.e., after needle and guidewire advancement but before sheath placement) to nick the skin and allow sheath entry. In the enhanced embodiment utilizing the integrated needle-dilator 14′, the same scalpel element 17 may be reconfigured with spring-loaded auto-retraction capability and repositioned in the sequence to operate earlier in the procedure, prior to needle advancement rather than after guidewire placement.

The lancet mechanism may be adjusted for optimal puncture depth, force, and consistency through testing on synthetic skin and soft tissue (e.g., Syndaver) and validation on human cadaver tissue. The auto-retracting design ensures that the lancet does not interfere with subsequent advancement of the integrated needle-dilator 14′ and guidewire 16.

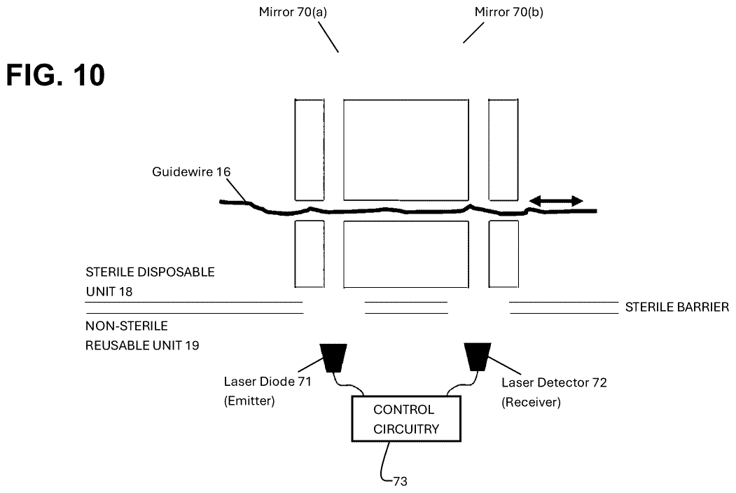

Further with respect to certain exemplary embodiments, novel optical detection system 70, illustrated in FIG. 10, provides precise tracking of guidewire position and advancement. This system addresses a critical limitation in existing automated vascular access devices by confirming that the guidewire 16 has successfully emerged from the needle tip and advanced to the desired depth within the vessel.

The optical detection system 70 comprises one or more laser diodes 71 positioned in the non-sterile reusable control unit 19 that emit narrow-diameter laser beams (approximately matching the guidewire diameter of 0.014″ to 0.035″) across the guidewire path. Photocell detectors 72 are positioned in the control unit 19 to receive the laser beams. When the guidewire 16 is not present in the beam path, the photocell detector 72 produces a first signal indicating light detection. When the guidewire 16 interrupts the laser beam path, the photocell 72 produces a second signal indicating light occlusion, thereby detecting guidewire presence and position.

Optionally, multiple optical detectors 71, 72 may be arranged in series along the guidewire path within the sterile detection zone. Each detector corresponds to a specific distance of guidewire advancement. By monitoring which detectors have been interrupted in sequence, the device precisely determines the length of guidewire that has advanced beyond the needle tip (typically 10-15 cm for effective cannulation).

A key innovation of the optical detection system 70 is the use of mirrors 70a, 70b to maintain sterility of the guidewire 16 and disposable components 18 while positioning the electronic components (laser source 71, photocell detectors 72, control circuits 73) in the non-sterile reusable base unit 19. As shown in FIG. 10, small mirrors 70a, 70b positioned within the sterile disposable cassette 18 reflect the laser beam from the non-sterile control unit 19 across the guidewire path in the sterile detection zone 74 and back to the photocell detector 72 in the control unit 19. The laser beam path crosses from the non-sterile environment to the sterile environment (containing the guidewire 16) only via the optical reflection off the mirrors 70a, 70b, avoiding any electrical or mechanical interface that would compromise sterility.

This optical detection system 70 may provide one or more of the following advantages: (1) confirmation that the guidewire 16 has successfully emerged from the needle tip without obstruction; (2) precise measurement of guidewire advancement distance; (3) early detection of guidewire advancement failure, allowing the operator to make adjustments (e.g., lower needle angle, slight withdrawal) before proceeding; and (4) maintenance of sterility for single-use disposable components while minimizing their cost and complexity.

Building upon the pressure transducers 20 and 21 described in the basic device configuration, the enhanced embodiment incorporates additional sensing modalities within the custom needle hub. First, micro-electromechanical systems (MEMS) pressure transducers may provide miniaturized, highly sensitive pressure sensing to detect the characteristic pressure waveforms indicating arterial or venous puncture. Second, bioimpedance sensors within the needle hub may detect changes in electrical impedance as the needle tip transitions from tissue to blood vessel lumen, providing additional confirmation of successful vessel entry. Third, optical sensors (distinct from the optical guidewire detection system 70 described above) within the needle hub may detect the “flash” of blood entering the needle lumen upon vessel puncture through changes in light absorption or reflection.

Such a multi-modal sensing approach provides redundant confirmation of successful vessel puncture and type (arterial vs. venous), increasing procedural reliability compared to systems relying on a single sensing modality.

The AVAD may include a plurality of mechanisms and electronic circuits configured to drive/operate the components 11-21, 60-74, and 14′. The mechanisms and electronic circuits may be incorporated in the device head 10 and the body 30. For example, the AVAD may include: a mechanism for driving the needle 12 and for injecting the Doppler gel and the antiseptic; a mechanism for driving the needle 13 and for injecting anesthetic; a mechanism for driving the arterial needle 14 or integrated needle-dilator 14′; a mechanism for driving the arterial sheath 15; a mechanism for driving the wire 16 (e.g., capstan wire drive as shown in FIG. 4(a)); a mechanism for driving/operating the scalpel 17 (or auto-retracting lancet configuration); a linear servo actuator 64 for adjusting needle angle from 30° to 70°; and control circuitry 73 for the optical guidewire detection system 70. The AVAD may further include mechanisms/electronic elements for converting the operator's actions on the trigger 40 (e.g. operator pushes the trigger 40) to operations of the components (e.g., pushing the trigger may cause the needle 13 to exit from the unit 18 and advance such as to penetrate the skin of the patient; pushing the trigger may sequentially activate each step of the method for automatically placing the vascular sheath).

Further, the AVAD may include a plurality of electronic devices, microprocessors and memories. The electronic devices may be configured to receive signals from the Doppler sensor 11, the pressure transducers 20 and 21, the bioimpedance sensors, the optical blood detection sensors, the MEMS pressure transducers, the optical guidewire detection system 70, and the ultrasound unit 60 API. The microprocessors and memories are configured to process signals and data received from the electronic devices and to display on the display screen 50 (which may be an LCD screen, tablet, or smartphone screen, or any other means of visual displaying, or audio signaling the operator) information about the status of the AVAD, the ultrasound image with superimposed crosshair trajectory guidance 62, and confirmation of successful vessel puncture and guidewire advancement.

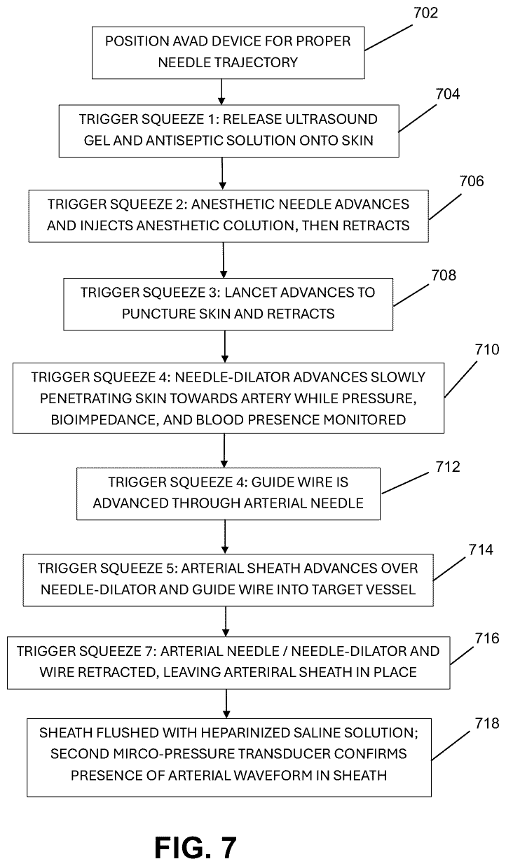

The method of operating the AVAD device (such as to perform the automatic placement of the vascular sheath) and the functioning of the AVAD is explained hereinafter according to an exemplary embodiment of the invention and with reference to FIG. 7 and the enhanced features described above. In a stand-by mode (before beginning the medical procedure of placing the vascular sheath) the components 12-17 are all hidden inside a corresponding port of the device head 10. The trigger 40 (similar to the trigger of a drill) may be used to sequentially activate each step of the methods/procedures described hereinafter. A display screen 50 may indicate the status of the device and the steps/functions of the medical procedure, and display the real-time ultrasound image with trajectory guidance crosshair 62.

At step 702, the operator initially holds the AVAD device over the patient's target region (e.g., neck for internal jugular vein, groin for femoral vessels) and activates the integrated ultrasound unit 60 to visualize the target vessel. The operator uses the color-flow Doppler feature to identify and confirm the target vessel type (arterial vs. venous). Using the thumb control 63, the operator adjusts the trajectory guidance crosshair 62 to center it over the target vessel on the ultrasound image displayed on screen 50. The device automatically tilts the needle carriage 65 via linear servo actuator 64 to the corresponding angle (30°-70°) to align with the selected trajectory.

At step 704, the operator squeezes the trigger 40 to release, via the needle tip 12, a mixture of ultrasound gel and antiseptic solution onto the skin. Then at step 706, the operator squeezes the trigger again, and a needle 13 advances and injects anesthetic solution and retracts.

In embodiments utilizing an integrated needle-dilator 14′ with enhanced auto-retracting lancet mechanism, the operator then at step 708 squeezes the trigger 40 again, and the scalpel/lancet 17 (configured as an auto-retracting spring-loaded lancet) rapidly advances to puncture approximately 3 mm of full-thickness skin, then automatically retracts.

Next at step 710, the operator squeezes the trigger 40 again and the integrated needle-dilator 14′ (or arterial entry needle 14 in embodiments without the integrated component) advances slowly from the disposable unit 18 at the pre-selected angle toward the target vessel. The integrated needle-dilator 14′ or needle 14 may include a micro-pressure transducer (e.g., MEMS-based), bioimpedance sensor, and optical blood detection sensor that are monitored by the control unit 19. The ultrasound image provides real-time visual confirmation of needle advancement toward the target vessel. When the sensors and/or ultrasound image confirm successful vessel puncture, the device signals the operator (via display screen 50, audible alarm, or haptic feedback) to proceed.

The operator squeezes the trigger 40 again at step 712, and a guide wire 16 in the disposable unit 18 is advanced through the needle 14 or integrated needle-dilator 14′ by a dual capstan drive system (FIG. 4(a)). The optical guidewire detection system 70 monitors guidewire advancement, with photocell detectors 72 detecting interruption of laser beams 71 reflected by mirrors 70a, 70b, thereby confirming emergence from the needle tip and tracking advancement distance. When the optical detection system 70 confirms that the wire 16 has advanced the target distance (typically 10-15 cm), the operator is notified by display screen 50 to squeeze the trigger 40 again.

In embodiments utilizing an integrated needle-dilator 14′, at step 714 the sheath 15 advances directly over the integrated needle-dilator 14′ and guidewire 16. In embodiments with separate needle and dilator components, the trigger 40 squeeze causes the scalpel blade 17 to advance along the needle 14 to nick the skin to allow sheath entry, and then retract, and a subsequent trigger squeeze then causes the arterial sheath 15 and dilator to advance over the needle 14 and wire 16 into the vessel.

Next at step 716, another trigger squeeze causes the needle 14 (or integrated needle-dilator 14′), wire 16, and dilator (if separate) to retract, leaving the sheath 15 in place. The sheath 15 is then automatically flushed at step 718 with heparinized saline solution and the second pressure transducer 21 (FIG. 4(a)) confirms the presence of the appropriate pressure waveform (arterial or venous) in the sheath to the operator via the display screen 50.

The separation of the control unit 19 from the disposable unit 18 keeps the “per use” cost of this device low, which will increase market opportunity. Complex and expensive components (ultrasound bracket 60a, optical detection electronics 70, linear servo actuator 64, driver motors, control circuitry 73, display panels 50) are in the reusable control unit 19. The self-contained nature of the device (incorporating ultrasound imaging 60, optical detection 70, gel, anesthesia, scalpel/lancet 17, etc.) and the ability to power it with rechargeable batteries makes it potentially appealing for placement in ambulances, emergency rooms and other pre-hospital settings, and allows non-physicians to perform this procedure safely. This device can be further configured for military use in battlefield, transport and pre-hospital settings.

While the exemplary embodiments have been shown and described, it will be understood by those skilled in the art that various changes in form and details may be made thereto without departing from the spirit and scope of the present disclosure as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. An automated vascular access device, comprising:
a body including a handle;
a device head attached to said body;
an ultrasound unit integrated with said device head, said ultrasound unit configured to generate real-time ultrasound images;
a display screen configured to display said ultrasound images;
a trajectory guidance system configured to superimpose a crosshair on said ultrasound images displayed on said display screen and to indicate a predicted needle trajectory;
an operator control configured to allow adjustment of said crosshair position on said display screen;
a needle carriage configured to hold a needle;
an actuator engaging said needle carriage and configured to adjust an angle of said needle carriage responsive to said crosshair position; and
a manually operable trigger configured to sequentially activate advancement of said needle toward a target vessel;
wherein said actuator automatically tilts said needle carriage to align said needle trajectory with said crosshair position in a plane of said ultrasound images, such that upon activation of said trigger, said needle advances at an angle corresponding to said crosshair position.

2. The automated vascular access device of claim 1, wherein said ultrasound unit includes color-flow Doppler visualization configured to visually distinguish arteries from veins.

3. The automated vascular access device of claim 1, wherein said operator control comprises a thumb control positioned on said handle.

4. The automated vascular access device of claim 1, wherein said actuator comprises a linear servo actuator with real-time positional feedback configured to adjust said needle carriage angle through a range of 30° to 70° with respect to a plane of a patient's skin.

5. The automated vascular access device of claim 1, further comprising a skin puncture element positioned at said device head, said skin puncture element comprising a spring-loaded blade configured to puncture approximately 3 mm of full-thickness skin and automatically retract prior to advancement of said needle.

6. The automated vascular access device of claim 1, further comprising a needle hub attached to said needle, said needle hub comprising a plurality of sensors selected from the group consisting of MEMS-based hydrostatic pressure transducers, bioimpedance sensors, and optical blood detection sensors.

7. An automated vascular access device, comprising:
a body including a handle;
a device head attached to said body, said device head comprising a disposable unit and a reusable control unit configured to detachably connect to the device;
a needle-dilator positioned within said disposable unit, said needle-dilator comprising:
a proximal end and a distal end;
an obliquely-cut tip at said distal end configured to puncture a patient's skin and blood vessel;
a lumen extending from said proximal end to said distal end configured to receive a guidewire; and
variable stiffness elements providing variable stiffness of said needle-dilator along a length of said needle-dilator;
an optical guidewire detection system comprising:
at least one laser source positioned in said reusable control unit;
at least one photocell detector positioned in said reusable control unit; and
at least two mirrors positioned in said disposable unit configured to reflect a laser beam from said laser source across a guidewire path and to said photocell detector;
wherein said photocell detector is configured to produce a first signal when said guidewire is not interrupting said laser beam and a second signal when said guidewire interrupts said laser beam, thereby detecting presence and position of said guidewire; and
a manually operable trigger configured to sequentially activate advancement of said needle-dilator and said guidewire.

8. The automated vascular access device of claim 7, wherein said variable stiffness elements are selected from the group consisting of: scalloped indentations on one side of said integrated needle-dilator; relaxing incisions orthogonal to a long axis of said integrated needle-dilator; and combinations thereof.

9. The automated vascular access device of claim 8, wherein said variable stiffness elements comprise scalloped indentations having semi-circular profiles formed along a portion of said integrated needle-dilator length.

10. The automated vascular access device of claim 9, wherein said variable stiffness elements further comprise relaxing incisions on a side of said needle-dilator opposite said scalloped indentations.

11. The automated vascular access device of claim 7, wherein said needle-dilator is formed from polyetheretherketone (PEEK).

12. The automated vascular access device of claim 7, wherein said needle-dilator is stiffened by a central steel needle during the puncture phase, and softened by constraining the needle and advancing only the dilator and sheath during the cannulation phase.

13. The automated vascular access device of claim 7, wherein said optical guidewire detection system further comprises a plurality of optical detectors arranged in series along said guidewire path, each optical detector configured to detect guidewire presence at a different position along said guidewire path, thereby determining a length of guidewire advancement.

14. A method for performing automated vascular access with ultrasound guidance, comprising:
providing an automated vascular access device comprising:
a body including a handle;
a device head attached to said body;
an ultrasound unit configured to generate real-time ultrasound images with color-flow Doppler imaging;
a display screen configured to display said ultrasound images;
a trajectory guidance system configured to superimpose a crosshair on said ultrasound images;
an operator control configured to adjust said crosshair position;
a needle carriage configured to hold a needle; and
an actuator configured to adjust an angle of said needle carriage responsive to said crosshair position;
positioning said device head over a target region of a patient;
activating said ultrasound unit to visualize a target blood vessel;
using said color-flow Doppler imaging to identify and distinguish whether said target vessel is an artery or vein;
adjusting said crosshair position using said operator control to center said crosshair over said target vessel on said ultrasound images;
causing said actuator mechanism to automatically tilt said needle carriage to align a needle trajectory with said crosshair position;
activating a trigger to advance said needle at an angle corresponding to said crosshair position toward said target vessel;
monitoring said ultrasound images to confirm needle advancement toward said target vessel;
detecting vessel puncture using at least one sensor selected from the group consisting of:
pressure sensors, bioimpedance sensors, and optical blood detection sensors;
advancing a guidewire through said needle into said target vessel;
detecting guidewire position and advancement distance using an optical guidewire detection system;
advancing a sheath over said needle and guidewire into said target vessel; and
retracting said needle and guidewire while leaving said sheath positioned within said target vessel.

15. The method of claim 14, wherein said operator control comprises a thumb control, and said step of adjusting said crosshair position comprises moving said thumb control to reposition said crosshair up or down on said display screen.

16. The method of claim 14, wherein said step of activating a trigger to advance said needle further comprises activating a skin puncture element to pre-puncture approximately 3mm of full-thickness skin prior to needle advancement.

17. The method of claim 14, wherein said step of detecting guidewire position and advancement distance comprises:
interrupting at least one laser beam with said guidewire;
detecting said interruption with at least one photocell detector;
determining that said guidewire has successfully emerged from a tip of said needle; and
confirming that said guidewire has advanced a target distance beyond said needle tip.

18. The method of claim 14, wherein said needle comprises a needle-dilator having variable stiffness elements, and said step of advancing a sheath comprises advancing said sheath over said integrated needle-dilator without requiring a separate dilator component.

19. The method of claim 14, wherein said step of detecting vessel puncture comprises receiving signals from one or more sensors selected from the group consisting of: a MEMS-based hydrostatic pressure transducer configured to detect pressure waveforms; a bioimpedance sensor configured to detect impedance changes; an optical sensor configured to detect blood flash; and combinations of the foregoing.
